(19) United States
(12) Patent Application Publication
(10) Pub. No.: US 2016/0038714 A1
(43) Pub. Date: Feb. 11, 2016

(54) SYSTEM AND METHOD FOR DELIVERING CANCER TREATING AGENTS TO AN ORGAN SUCH AS THE BLADDER

(71) Applicant: JAMES G. GETSAY, ZELIENOPLE, PA (US)

(72) Inventor: JAMES G. GETSAY, ZELIENOPLE, PA (US)

(21) Appl. No.: 14/818,591

(22) Filed: Aug. 5, 2015

Related U.S. Application Data
(60) Provisional application No. 62/034,918, filed on Aug. 8, 2014.

(51) Int. Cl. A61M 25/00; A61M 25/10

(52) U.S. Cl. CPC A61M 25/003; A61M 25/0028; A61M 25/10; A61M 2205/3303; A61M 2025/0002; A61M 2025/1052; A61M 2025/0037; A61M 25/09

(57) ABSTRACT

A catheter for delivering, a hyperthermic liquid to the interior of an organ of a patient includes an inflow lumen structured to be inserted into the organ and deliver the hyperthermic liquid to the interior of the organ, and an outflow lumen structured to be inserted into the organ and evacuate the hyperthermic liquid from the interior of the organ. The catheter includes a proximal portion that is structured to be received within the internal tissue structure of the patient, wherein at least a portion of the inflow lumen and the outflow lumen are insulated in a manner wherein a temperature on an outside surface of the proximal portion of the catheter will not be more than one degree Fahrenheit above normal body temperature when the hyperthermic liquid is passed through the catheter.

### CROSS-REFERENCE TO RELATED APPLICATIONS

This application claims priority under 35 U.S.C. §119(e) from U.S. provisional patent application No. 62/034,918, entitled “System and Method for Delivering Cancer Treating Agents to an Organ such as the Bladder” and filed on Aug. 8, 2014, the contents of which are incorporated herein by reference.

### BACKGROUND OF THE INVENTION

1. Field of the Invention

The present invention pertains to the treatment of cancer, such as, without limitation, bladder cancer, including intravesical treatment of bladder cancer, and, in particular, to a system and method for delivering cancer treating agents (e.g., chemotherapeutic and immunotherapeutic agents) to the an organ such as the bladder.

2. Description of the Related Art

Each year multiple thousands of people are diagnosed with bladder cancer. Furthermore, bladder cancer remains one of the most expensive malignancies worldwide in terms of the cost incurred in making a diagnosis and in its subsequent treatment, due in large part to the diagnostic tests and treatments (surgical and non-surgical) patients are required to undergo in the management of this disease.

Roughly 70% of new bladder cancer cases are what is known as transitional cell bladder cancer, also referred to as superficial bladder cancer, wherein the cancer has not penetrated the bladder wall. Patients with superficial bladder cancer are often at increased risk of developing subsequent tumors such that in many cases, multiple surgeries and/or chemotherapy and/or immunotherapy treatments are required. Also, the recurrence rate for bladder cancer is estimated to be between 50% and 70%, with as much as fifteen years between the initial diagnosis and the second occurrence.

The standard of care for transitional cell bladder cancer is a treatment known as intravesical therapy. In intravesical therapy, the doctor uses a catheter to deliver a liquid agent, which may be a chemotherapy drug (such as, without limitation, mitomycin) or an immunotherapy agent (such as without limitation, Bacillus Calmette-Guearin (BCG) bacterium) directly into the bladder. Alter delivery of the agent, the catheter is removed and the patient is instructed to not urinate for one or two or even several hours to give the agent time to act (the patient may be sent home during this period). After sufficient time to allow the agent to act has passed, the agent is voided out by the patient.

Intravesical therapy as just described is more than 20 years old. It is evident that this mode of delivery is quite crude and raises multiple concerns including, without limitation, whether the agent is being delivered to the entire bladder, and whether the agent is being delivered under less than optimal conditions such that the chance of treatment success is decreased.

### SUMMARY OF THE INVENTION

In one embodiment, a catheter for delivering as hyperthermic liquid to the interior of an organ of a patient is provided that includes an inflow lumen structured to be inserted into the organ and deliver the hyperthermic liquid to the interior of the organ, and an outflow lumen structured to be inserted into the organ and evacuate the hyperthermic liquid from the interior of the organ. The catheter includes a proximal portion that is structured to be received within the internal tissue structure of the patient, wherein at least a portion of the inflow lumen and the outflow lumen are insulated in a manner wherein a temperature on art outside surface of the proximal portion of the catheter will not be more than one degree Fahrenheit above normal body temperature when the hyperthermic liquid is passed through the catheter.

In another embodiment, a method of delivering a hyperthermic treatment agent to an organ of a patient is provided. The method includes inserting a catheter into the organ of the patient through and internal tissue structure of the patient, flowing a hyperthermic liquid into and out of the organ through the catheter to raise a temperature within the organ, measuring the temperature within the organ and determining that the temperature within the organ has reached at least a predetermined level, and after determining that the temperature within the organ has reached at least the predetermined level, flowing the hyperthermic treatment agent into the organ through the catheter.

### BRIEF DESCRIPTION OF THE DRAWINGS

FIG. 1 is a schematic diagram of an infusion system 2 for delivering hyperthermic and/or normothermic agents to the entire internal surface of a patient's bladder according, to an exemplary, non-limiting embodiment of the present invention;

FIG. 2 is a block diagram of a patient monitoring component forming part of the infusion system of FIG. 1 according to one exemplary embodiment;

FIG. 3 is a flowchart illustrating a method of operation of and use of the infusion system of FIG. 1 to provide cancer treatment to a patient according to a first exemplary, non-limiting embodiment; and

FIG. 4 is a flowchart illustrating a method of operation of and use of the infusion system of FIG. 1 to provide cancer treatment to a patient according to a second exemplary, non-limiting embodiment.

### DETAILED DESCRIPTION OF EXEMPLARY EMBODIMENTS

As used herein, the singular form of “a”, “an”, and “the” include plural references unless the context clearly dictates otherwise.

As used herein, the statement that two or more parts or elements are “coupled” shall mean that the parts are joined or operate together either directly or indirectly, i.e., through one or more intermediate parts or elements, so long as a link occurs.

As used herein, “directly coupled” means that two elements are directly in contact with each other.

As used herein, “fixedly coupled” or “fixed” means that two elements are coupled so as to move as one while maintaining a constant orientation relative to each other.

As used herein, the word “unitary” means a part is created as a single piece or unit. That is, a part that includes pieces that are created separately and then coupled together as a unit is not a “unitary” part or body.

As employed herein, the statement that two or more parts or elements “engage” one another shall mean that the parts exert a force against one another either directly or through one or more intermediate parts or elements.

As employed herein, the term “number” shall mean one or an integer greater than one (i.e., a plurality).

As used herein, the term “component” is intended to refer to a computer related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

As used herein, the term “normal body temperature” shall mean 98.6° Fahrenheit.

As used herein, the term “hyperthermic” shall mean above normal body temperature.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

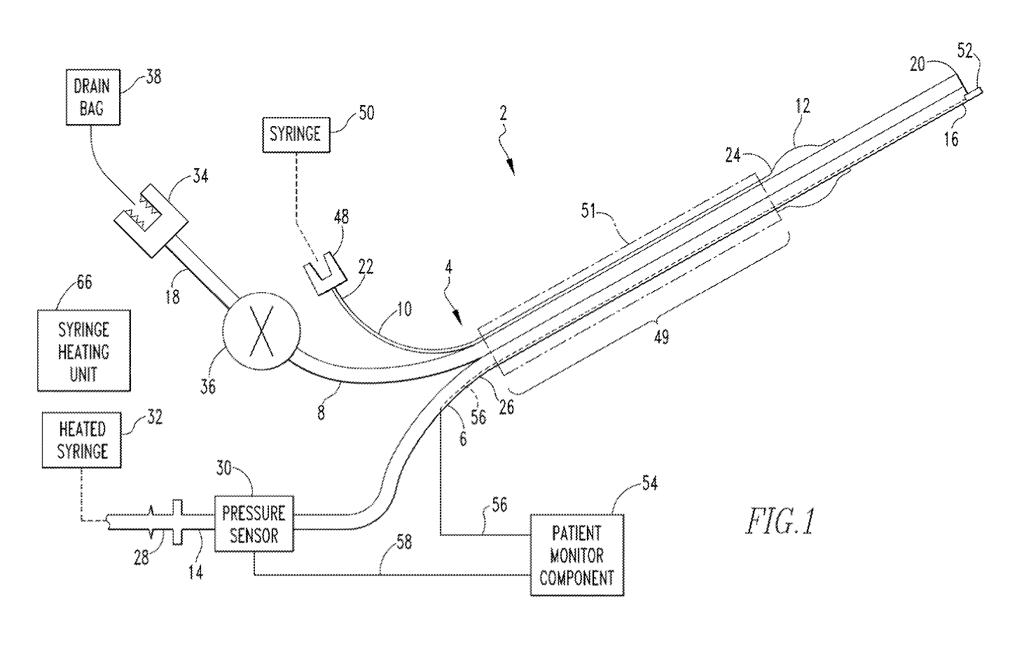

FIG. 1 is a schematic diagram of an infusion system 2 for delivering hyperthermic and/or normothermic agents to the entire internal surface of a patient's bladder according to an exemplary, non-limiting embodiment of the present invention. infusion system 2 includes a catheter 4 that is structured to be inserted into the bladder through the patient's urethra to allow selective delivery of and evacuation of fluids, such as hyperthermic chemotherapeutic and/or immunotherapeutic agents, to horn the patient's bladder for purposes of intravesical therapy. As described in greater detail herein, infusion system 2 further includes components that are structured to monitor and report the temperature inside the patient's bladder and the pressure of the fluid being delivered to the patient's bladder. The significance of these features is described in detail elsewhere herein.

As seen in FIG. 1, catheter 4 includes three lumens that are coupled to one another. More specifically, catheter 4 includes an inflow lumen 6, an outflow lumen 8, and a balloon lumen 10. Inflow lumen 6 is a tubular member having a first end 14 and a second, opposite end 16, and is made of for example, and without limitation, a rubber, silicone, or latex material. As described in greater detail herein, inflow lumen 6 is structured to enable fluids to be delivered therethrough to the interior of the bladder of the patient. Outflow lumen 6 is also a tubular member having a first end 18 and a second, opposite end 20, and is made of for example. and without limitation, a rubber, silicone, or latex material. As described in greater detail herein, outflow lumen 8 is structured to enable fluids to be evacuated therethrough from the interior of the bladder of the patient. Balloon lumen 10 is a tubular member having a first end 22 and a second, opposite end 24. Balloon lumen 10 also includes an inflatable sealing balloon 12 coupled to the second end 24 thereof. As described in greater detail herein, balloon lumen 10 is structured to enable sealing balloon 12 to be selectively inflated by flowing, a fluid, such as air, through balloon lumen 10, in order to seal off the interior of the bladder during treatment. In the exemplary embodiment, and as seen in FIG. 1, the portions of inflow lumen 6, outflow lumen 8, and balloon lumen 10 beginning at a central position 26 of catheter 4 and extending to the second ends 16, 20 and 24 of inflow lumen 6, outflow lumen 8, and balloon lumen 10, respectively, are held together and attached to one another by a suitable adhesive material. hi addition, as in FIG. 1, the remaining portions of inflow lumen 6 outflow lumen 8, and balloon lumen 10 extending from central position 26 to the first and 14, 18, 22 of inflow lumen 6, outflow lumen 8, and balloon lumen 10, respectively, are not attached to one another, but instead are separate from and able to move freely relative to one another.

First end 14 of inflow lumen 6 includes a male Luer fitting 28 and a pressure sensor 30. Male Luer fitting 28 is structured to enable a syringe, such as heated syringe 32 (i.e. a syringe holding a heated liquid), to be coupled thereto for injecting a fluid, such as heated saline, a hyperthermic chemotherapeutic agent or a hyperthermic immunotherapeutic agent, into inflow lumen 6. Pressure sensor 30 is provided in line with inflow lumen 6 and is structured to measure the pressure of the fluid passing through inflow lumen 6 and thus the pressure within the patient's bladder). First end 18 of outflow lumen 8 includes a female Luer fitting 34 and an outflow shut off valve 36. Female Luer fitting 34 is structured to enable a drain bag 38 to be coupled thereto for receiving, fluids that are evacuated through outflow lumen 8 as described herein. First end 22 of balloon lumen 10 includes a female Toomey fitting 48 that is structured to enable a syringe 50 to be coupled to balloon lumen 10 for injecting air into and withdrawing air from balloon lumen 10 in order to selectively inflate and deflate sealing balloon 12.

As seen in FIG. 1, in the illustrated embodiment, a portion 49 of catheter 4 extending from about central position 26 to the bottom of sealing balloon 12 is encapsulated in an insulating material 51. The insulating material a be any suitable, bio-compatible material that provides a sufficient insulation function, such as, without limitation, a latex material or a urethane material. The purpose and function of insulating material 51 is to insulate portion 49, which is the portion of catheter 4 that will rest within the patient's urethra when catheter 4 is in use, and thereby prevent harm or damage to the urethra as a result of the heat of the hyperthermic liquids passing through catheter 4. More specifically, in the exemplary embodiment, the material of insulating material 51 is chosen such that the temperature of the external surface of catheter 2 in the portion 49 (i.e., the external surface of the insulating material 51) will be no more than one degree Fahrenheit above normal body temperature (i.e., the temperature will be between 98.6° F. and 99.6° F., but no more than 99.6° F). This insulation function will protect the internal tissue of the urethra when hyperthermic fluids are being delivered through catheter 4 as described.

In an alternative embodiment, rather than providing insulating material 51 at portion 49, inflow lumen 6 and outflow lumen 8 may be manufactured from insulative materials that provide the insulator function just described. In such a configuration, the materials are chosen such that the external surfaces of inflow lumen 6 and outflow lumen 10 will be no more than one degree Fahrenheit above normal body temperature. As a result, the exterior surface of portion 49 will not experience an undesirable temperature increase.

In one exemplary embodiment, inflow lumen 6 and outflow lumen 10 are both made of polytetrafluoroethylene (PTFE), with each having an inside diameter of approximately 0.0625 inches and a wall thickness of approximately 0.020 inches. In addition, in this exemplary embodiment, insulating material 51 is a silicone elastomer and the outer diameter of portion 49 of catheter 4 is 16 French.

Catheter 4 further includes a temperature sensor 52 coupled to and provided at end 16 of inflow lumen 6. In the illustrated embodiment, temperature sensor 52 is directly coupled to the exterior of the and 16 of inflow lumen 6. In the exemplary embodiment, temperature sensor 52 is a thermistor, and more specifically a bead thermistor. Also in the exemplary embodiment temperature sensor 52 is positioned such that approximately half of the body thereof extends past the terminal end of catheter 4 at end 16 of inflow lumen 6. Temperature sensor 52 is structured to measure the temperature in the patient's bladder when catheter 4 is inserted therein. Temperature sensor 52. is coupled to a patient monitor component 54 (described below) forming a part of infusion system 2 b a wire 56 that extends down along a portion of the length of inflow lumen 6. Also coupled to patient monitor component 54 is pressure sensor 30 by way of a wire 58.

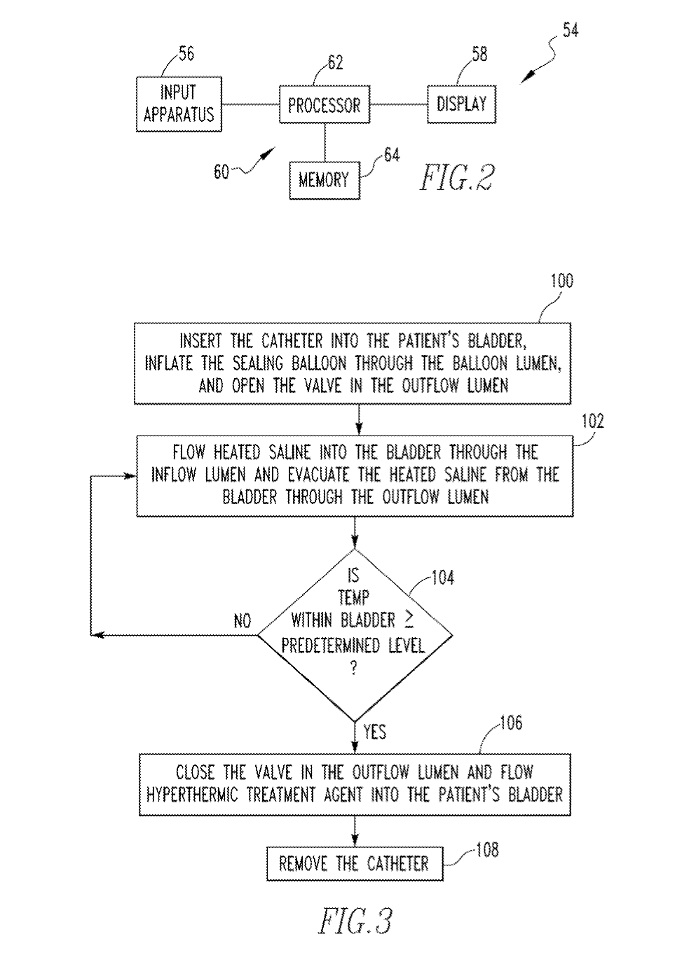

Patient monitor component 54 is a computing device that is structured to receive the measurements/readings from pressure sensor 30 and temperature sensor 52 and display such readings visually so that they may be monitored by a physician or other health-care provider. FIG. 2 is a block diagram of patient monitoring component 54 according to one exemplary embodiment As seen in FIG. 2, the exemplary patient monitoring component 54 includes an input apparatus 55 such as a keypad or touchscreen, a display 58, such as a number of LEDs or an LCD, and a processor apparatus 60. A user is able to provide input into processor apparatus 60 using input apparatus 56, and processor apparatus 60 provides output signals to display 5 to enable display 58 to display information to the user as described in detail herein (e.g., the pressures measured by pressure sensor 30 and or the temperatures measured by temperature sensor 52). Processor apparatus SO comprises a processor 62 and a memory 64. Processor 62 may be, for example and without limitation, a microprocessor (μP), a microcontroller, or some other suitable processing device, that interfaces with memory 64. Memory 64 can be any one or more of as variety of types of internal and/or external storage media such as, without limitation, RAM, ROM, EPROM(s), EEPROM(s), FLASH, and the like that provide a storage register, i.e., a machine readable medium, for data storage such as in the fashion of an internal storage area of a computer, and can be volatile memory or nonvolatile memory. Memory 64 has stored therein a number of routines that are executable by processor 62 to enable operation of infusion system 2 as described herein.

Finally, referring again to FIG. 1, in the exemplary embodiment, infusion system 2 includes a syringe heating unit 66 which is structured to hold and heat a number of syringes which each contain a liquid such as a chemotherapeutic agent or an immunotherapeutic agent, in order to bring the contents of the syringes up to hyperthermic temperatures for subsequent use as described herein.

FIG. 3 is a flowchart illustrating a method of operation of and use of infusion system 2 and catheter 4 to provide cancer treatment to a patient according to a first exemplary, non-limiting embodiment. The method begins at step 100, wherein catheter 4 is inserted into the patient's bladder through the patient's urethra. In the exemplary embodiment, this insertion is performed using a guide wire (not shown) that is first inserted into the patient's bladder through the urethra. Catheter 4 is then inserted into the patient's bladder through the patient's urethra by passing, the inflow lumen 6 along the guide wire. Once catheter 4 is in place, the guide wire is removed by pulling it out of the patient's urethra along the interior of inflow lumen 6. Following such insertion, portion 49 of catheter 4 will be located within the patient's urethra and the distal portion of catheter 4 forward of portion 49 will be located within the patient's bladder. Also at step 100, following such insertion, the sealing balloon 12 is inflated through balloon lumen 10 by attaching, syringe 50 to Toomey fitting 48 and injecting air into balloon lumen 10. Still also at step 100, the valve 36 in outflow lumen 8 is opened. Next, at step 102, heated saline is flowed into the patient's bladder through inflow lumen 6 and evacuated from the patient's bladder in a continuous manner through outflow lumen 8. This is accomplished by attaching one or more syringes containing saline (sequentially if multiple syringes are used to Luer fitting 28 through a tubing circuit that includes an in line heat exchanger (not shown), as described, in U.S. patent application Ser. No. 13/1998,367, entitled “fluid Infusion Device and Method”, the disclosure of which is incorporated, herein by reference. As a result, heated saline will be injected into inflow lumen 6. During this process because valve 36 is in an open position, the heated saline, once it reaches the patient's bladder through inflow lumen 6, will then be evacuated from the patient's bladder through outflow lumen 8 and into drain bag 38. Next, at step 104, a determination is made as to whether the temperature within the patient's bladder is greater than or equal to a predetermined level. This is done by taking a reading from temperature sensor 52. The predetermined level is a temperature that has been determined to be optimal for the treatment being performed, and in the exemplary embodiment is between 101° F. and 104° F., and in another exemplary embodiment is between 104° F. and 110° F. (e.g., 43°F.). If the answer at step 104 is no, meaning that the predetermined temperature level has not been reached, the method returns to step 102 wherein the flow of heated saline is continued in an effort to continue to bring up the temperature of the bladder. If, however, the answer at step 104 is yes, meaning that the temperature within the bladder has at least reached the predetermined level, then the flow of heated saline is stopped and the method proceeds to step 106. At step 106, valve 36 is closed and thereafter a hyperthermic treatment agent is flowed into the patient's bladder through inflow lumen 6. In particular, in this step, one or more heated syringes 32 containing a hyperthermic treatment agent, such as a hyperthermic chemotherapeutic agent or a hyperthermic immunotherapeutic agent, are coupled to Luer fitting 28 and the hyperthermic treatment agent is injected into inflow lumen 6. The amount of hyperthermic treatment agent that is injected in this step will be a predetermined amount that has been determined to be effective for the current treatment. Following step 106, at which point the bladder will be filled with the hyperthermic treatment agent, catheter 4 is removed from the patient. The patient is then instructed to wait a certain period of time to enable the hyperthermic treatment agent act, after which point the patient voids the hyperthermic treatment agent.

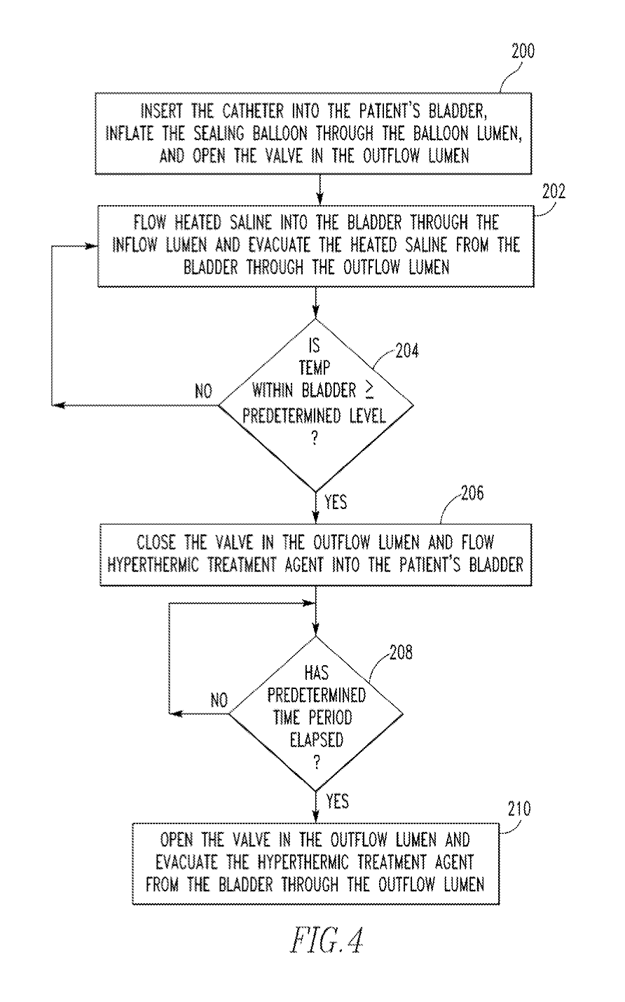

FIG. 4 is a flowchart illustrating a method of operation of and use of infusion system 2 and catheter 4 to provide, cancer treatment to a patient according to a second exemplary, non-limiting embodiment. The method begins at step 200, wherein catheter 4 is inserted into the patient's bladder through the patient's urethra. Following such insertion, portion 49 of catheter 4 will be located within the patient's urethra and the distal portion of catheter 4 forward of portion 49 will be located within the patient's bladder. Also at step 200, following such insertion, the sealing balloon 12 is inflated as described above. Still also at step 200, the valve 36 in outflow lumen 8 is opened. Next, at step 202, heated saline is flowed into the patient's bladder through inflow lumen 6 and evacuated from the patient's bladder in a continuous manner through outflow lumen 8 as described above. Then, at step 204, a determination is made as to whether the temperature within the patient's bladder is greater than or equal to a predetermined level based on a reading from temperature sensor 52. As noted above, the predetermined level is a temperature that has been determined to be optimal for the treatment being performed, and in the exemplary embodiment is between 101° F. and 104° F. If the answer at step 204 is no meaning that the bladder had not yet been brought up to the optimal temperature, the method returns to step 202 wherein the flow of heated saline is continued in an effort to continue to bring up the temperature of the bladder. If, however, the answer step 204 is yes, meaning that the optimal temperature within the bladder has been reached, then the flow of heated saline is stopped and the method proceeds to step 206. At step 206, valve 36 is closed and thereafter a hyperthermic treatment agent, such as a hyperthermic chemotherapeutic agent or a hyperthermic immunotherapeutic agent, is flowed into the patient's bladder through inflow lumen 6 using one or more heated syringes 32 as described above. Following step 206, at which point the bladder will be filled with the hyperthermic treatment agent, a determination has been made as to whether a predetermined time period has elapsed. In this embodiment, that predetermined time period is the period for which the hyperthermic treatment agent should remain within the patient's bladder for the treatment to be effective. If the answer at step 208 is no, then the method continues to wait until that time period has elapsed. If, however, the answer at step 208 is yes, then the method proceeds to step 210, wherein the valve 36 in the outflow lumen 8 is opened, thereby causing, the hyperthermic treatment agent to be evacuated from the bladder. Following such evacuation, the bladder may be purged using a saline solution delivered to and evacuated from the bladder using catheter 4. Furthermore, steps 202 through 210 may be repeated one or more additional times in order to introduce different hyperthermic treatment agents into the bladder as part of a treatment protocol. Once all treatments are completed, catheter 4 may then be removed from the patient.

In one particular, non-limiting embodiment, pressure measurements are made during steps 102 and 106 of FIG. 3 and steps 202 and 206 of FIG. 4, with such pressure measurements being continuously monitored and with appropriate adjustments to flow rate being made to ensure patient safety/comfort.

Thus, the system and method of drug delivery described herein in the various embodiments offers a number of advantages important to the implementation of more effective local therapies of bladder cancer. First, they allow the sequential administration of different elements of intravesical therapies within one overall procedure individually or in defined combination (for example, chemotherapeutic agents, small molecule inhibitors and modulators of inflammation, and biological agents). Second, they also facilitate the uniform and consistent exposure of tumor tissues to the selected concentrations of biologic and chemotherapeutic agents. Third, they allow the controlled and consistent use of local bladder hyperthermia to: a) enhance the tumor uptake of chemotherapeutic or biologic factors; b) modify the pattern of the resulting inflammatory response within the tumor tissues; and c) promote tumor entry of immune cells. Fourth, they allow for longitudinal monitoring of a) uptake of the applied therapeutic agents and b) the response to such agents, measured by the rates of local release of pro- and anti-inflammatory factors, allowing for personalized patient-specific adjustment of the composition and/or concentrations of the therapeutic factors, in order to assure their optimal effectiveness and to minimize the side-effects.

Furthermore, the treatment methods described herein can be conducted as an outpatient procedure, with minimal to no patient morbidity. The treatments are also minimally invasive, requiring simply the insertion of the catheter 4 before and then subsequent removal of the catheter 4 after the treatments. In addition, the treatment methods may potentially reduce treatment discomfort by allowing patients to rest comfortably during treatment periods rather than being asked to “hold in” a chemotherapeutic and/or immunotherapeutic agent. In this regard, it is believed that the treatment methods described herein will play an important role in enhancing the efficacy and tolerability of bladder cancer treatment for years to come.

Moreover, while the invention has been described herein in the various embodiments in connection with treatments for bladder cancer, it will be understood that the infusion system 2, catheter 4 and methods described herein may also be used to treat cancers of other body organs such as, without limitation, the kidneys.

In the claims, any reference signs placed between parentheses shall not be construed as limiting the claim The word “comprising” or “including” does not exclude the presence of elements or steps other than those listed in a claim. In a device claim enumerating several means, several of these means may he embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A catheter for delivering a hyperthermic liquid to an interior of an organ of a patient, comprising:
an inflow lumen structured to be inserted into the organ and deliver the hyperthermic liquid to the interior of the organ; and
an outflow lumen structured to be inserted into the organ and evacuate the hyperthermic liquid from the interior of the organ;
wherein the catheter includes a proximal portion that is structured to be received within an internal tissue structure of the patient, and wherein at least a portion of the inflow lumen and at least a portion of the outflow lumen are insulated in a manner wherein a temperature on an outside surface of the proximal portion of the catheter will not be more than one degree Fahrenheit above normal body temperature when the hyperthermic liquid is passed through the catheter.

2. The catheter according to claim 1, wherein the organ is the bladder and wherein the internal tissue structure of the patient is a urethra of the patient.

3. The catheter according to claim 1, wherein the hyperthermic liquid has a temperature that is at least one degree more than normal body temperature.

4. The catheter according to claim 3, wherein the temperature of the hyperthermic liquid is equal to or greater than 102° F.

5. The catheter according to claim 1, wherein the hyperthermic liquid is heated saline, a chemotherapeutic agent or an immunotherapeutic agent.

6. The catheter according to claim 1, wherein the catheter includes an insulative encapsulating material that surrounds the at least a portion of the inflow lumen and the at least a portion of the outflow lumen for insulating the inflow lumen and the outflow lumen in the proximal portion.

7. The catheter according to claim 6, wherein the insulative encapsulating material is a latex material or a urethane material.

8. The catheter according to claim 1, wherein the inflow lumen and the outflow lumen are each made from an insulative material such that the temperature on the outside surface of the proximal portion of the catheter will not be more than one degree above normal body temperature when the hyperthermic liquid is passed through the catheter.

9. The catheter according claim 1, wherein the catheter has a distal portion coupled to the proximal portion, wherein the distal portion is structured to be received within the organ, and wherein the distal portion includes a temperature sensor for sensing a temperature within the organ.

10. The catheter according to claim 9, wherein the temperature sensor is attached directly to the inflow lumen in the distal portion.

11. The catheter according to claim 9, wherein the temperature sensor is a thermistor.

12. The catheter according to claim 10, wherein the temperature sensor is a bead thermistor.

13. The catheter according to claim 11, wherein a portion of the bead thermistor extends beyond a terminal end of the distal portion.

14. The catheter according to claim 1, further comprising a pressure sensor coupled to the inflow lumen for sensing a fluid pressure being provided to the organ.

15. The catheter according to claim 14, wherein the pressure sensor is provided inline within the inflow lumen.

16. The catheter according to claim 1, further comprising a balloon lumen having an inflatable sealing balloon, the sealing balloon being structured to be received within the organ.

17. The catheter according to claim 1, wherein the outflow lumen includes a valve for controlling a flow of the hyperthermic liquid within the outflow lumen.

18. A system for delivering hyperthermic liquid to an organ of a patient, comprising a catheter including a temperature sensor according to claim 9 and a patient monitor component coupled to the temperature sensor, the patient monitor component being structured to display the temperature with the organ.

19. A system for delivering a hyperthermic liquid to an organ of a patient, comprising a catheter including a pressure sensor according to claim 14 and a patient monitor component coupled to the pressure sensor, the patient monitor component being structured to display the fluid pressure.

20. A method of delivering a hyperthermic treatment agent to an organ of a patient, comprising:
inserting a catheter into the organ of the patient through and internal tissue structure of the patient;
flowing a hyperthermic liquid into and out of the organ through the catheter to raise a temperature within the organ;
measuring the temperature within the organ and determining that the temperature within the organ has reached at least a predetermined level; and
after determining that the temperature within the organ has reached at least the predetermined level, flowing the hyperthermic treatment agent into the organ through the catheter.

21. The method according to claim 20, wherein the organ is a bladder of the patient and wherein the internal tissue structure is a urethra of the patient.

22. The method according to claim 20, wherein the hyperthermic liquid is heated saline.

23. The method according to claim 20, wherein the hyperthermic treatment agent is a chemotherapeutic agent or an immunotherapeutic agent.

24. The method according to claim 20, wherein the inserting causes a proximal portion of the catheter to be located within the internal tissue structure of the patient, and wherein the catheter is structured such that the flowing of the hyperthermic liquid and the flowing of the hyperthermic treatment agent will not cause a temperature of an external surface of the proximal portion to be more than one degree Fahrenheit above normal body temperature.

25. The method according to claim 20, further comprising causing the hyperthermic treatment agent to be evacuated from the organ through the catheter after the hyperthermic treatment agent has been inside the bladder for at least a predetermined period of time.

26. The method according to claim 25, wherein the hyperthermic treatment agent is a first hyperthermic treatment agent, the method further comprising flowing a second hyperthermic treatment agent different than the first hyperthermic treatment agent into the organ through the catheter after the first hyperthermic treatment agent has been evacuated from the organ and without removing the catheter from the patient between the evacuation of the first hyperthermic treatment agent and the flowing of the second hyperthermic treatment agent.

27. The method according to claim 20, wherein a distal portion of the catheter that is structured to be received within the organ includes a temperature sensor, and wherein the measuring the temperature within the bladder is performed using the temperature sensor.

28. The method according to claim 20, further comprising measuring a pressure within the organ and adjusting a flow rate of at least one of the flowing steps based on the measured pressure.
